(19) United States
(12) Patent Application Publication
(10) Pub. No.: US 2006/0045915 A1
(43) Pub. Date: Mar. 2, 2006

(54) Odor eliminating foot powder composition

(76) Inventor: Diane Stess-Kirschner, Tamarac, FL (US)

Correspondence Address: Robert M. Downey;ROBERT M. DOWNEY, P.A., Suite 300, 601 South Federal Highway, Boca Raton, FL, 33432, US

(21) Appl. No.: 10/930,564

(22) Filed: Aug. 31, 2004

(51) Int. Cl. A61K 8/27; A61K 9/14

(52) U.S. Cl. 424/489; 424/67

(57) ABSTRACT

A powder composition for eliminating foul odors associated with bromhidrosis and hyperhidrosis includes aluminum potassium sulfate in an amount of between 22% and 62% by weight, zinc oxide in an amount of between 8% and 36% by weight, and zinc undecylenate in an amount of between 6% and 34% by weight. In various embodiments, the composition may further include talc, corn starch, Kaolin and/or silicone dioxide. The powder composition is used in footwear and prosthetic fittings to eliminate bacteria and inhibit further bacterial growth that causes odors.

### BACKGROUND OF THE INVENTION

1. Field of the Invention

The present invention relates to a powder composition and, more particularly, to an odor eliminating powder composition for application to prosthetic fittings and attachments and within footwear to prevent odors associated with bromhidrosis and hyperhidrosis.

2. Discussion of the Related Art

People who seem to have a chronic problem with unpleasant foot odor or stinky feet, may be suffering from a condition known as bromhidrosis. A primary cause of bromhidrosis is excessive perspiration or hyperhidrosis, which provides a breeding ground for bacteria and yeast. As cellular debris from the bacteria and yeast begin to break down, a foul odor begins to eminate from the feet. This foul odor, along with the sweat, bacteria and yeast causing the odor, remain in footwear after removal from the feet. Similar foul odors develop in prosthetic fittings and attachments as sweat and moisture cause fungi and bacteria to grow in the sockets and fittings where skin tissue engages the prosthetic.

Presently, there are numerous non-prescription (off the shelf) powder products available on the market for treating foot odor. These products use strong fragrances which have the effect of masking the foul odor emanating from the feet and footwear. As a result, the desired effect of these products is not long lasting and, often, the resultant odor from the combination of the fragrance and stinky feet is equally unpleasant.

Accordingly, there remains a definite need in the art for a foot powder composition which can be applied to the feet, footwear as well as prosthetic attachments and fittings for eliminating foul odors. Moreover, there is an urgent need for a foot powder composition which eliminates foul odors in footwear and prosthetic attachments, rather than simply masking these odors. Furthermore, there is a need for an odor eliminating powder composition which eliminates bacteria in footwear and prosthetic fittings and inhibits further bacterial growth which causes foul odors.

### SUMMARY OF THE INVENTION

The present invention is directed to a powder composition for eliminating foul odors associated with bromhidrosis and hyperhidrosis. The composition includes aluminum potassium sulfate in an amount of between 22% and 62% by weight, zinc oxide in an amount of between 8% and 36% by weight, and zinc undecylenate in an amount of between 6% and 34% by weight. In various embodiments, the composition may further include talc, corn starch, Kaolin and/or silicone dioxide. The powder composition is used in footwear and prosthetic fittings to eliminate bacteria and inhibit further bacterial growth that causes odors.

The powder composition is sprinkled into footwear such as tennis shoes, sneakers, bowling shoes, ballet shoes, boots, boat shoes as well as dress shoes to eliminate foul odors. The powder composition can also be used by sprinkling within the sockets and other areas of prosthetic attachments and fittings which contact the skin, thereby inhibiting bacterial growth and preventing odors. In use, the powder composition is sprinkled in each shoe or the prosthetic once a day for seven days. Thereafter, the powder composition need only be applied once a week.

### DETAILED DESCRIPTION OF THE PREFERRED EMBODIMENT

The present invention is directed to an odor eliminating powder composition for use in footwear and prosthetic fittings and attachments.

The odor eliminating powder composition of the present invention includes the following essential ingredients: aluminum potassium sulfate; zinc oxide; and zinc undecylenate. Other non-essential ingredients of the composition include: Kaolin; silicone dioxide; talc; and corn starch.

In a preferred embodiment, aluminum potassium sulfate is present in the composition in an amount of between 22% and 62% by weight of the composition. Zinc oxide is present in the composition in an amount of between 8% and 36% by weight of the composition. Zinc undecylenate is present in the composition in an amount of between 6% and 34% by weight of the composition.

Any combination of one or more of the non-essential ingredients may be present with the above-identified essential ingredients. When used, Kaolin is present in the composition in an amount of between 2% and 28% by weight of the composition. Silicone dioxide, when used, is present in the composition in an amount of between 0.5% and 15% by weight of the composition. Talc may be present in the composition in an amount of between 1% and 18% by weight of the composition. Corn starch may be present in the composition in an amount of between 1% and 18% by weight of the composition.

The following are examples of formulas of the Odor Eliminating Powder Composition of the present invention, in accordance with various preferred embodiments thereof:

EXAMPLE 1

| | Amount |
|---|---|
| Ingredient | (% by Weight of the Composition) |
| Aluminum Potassium Sulfate | 42% |
| Zinc Oxide | 22% |
| Zinc Undecylenate | 20% |
| Kaolin | 15% |
| Silicone Dioxide | 1% |

EXAMPLE 2

| | Amount |
|---|---|
| Ingredient | (% by Weight of the Composition) |
| Aluminum Potassium Sulfate | 38% |
| Zinc Oxide | 18% |
| Zinc Undecylenate | 20% |
| Talc | 12% |
| Kaolin | 8% |
| Silicone Dioxide | 4% |

EXAMPLE 3

| | Amount |
|---|---|
| Ingredient | (% by Weight of the Composition) |
| Aluminum Potassium Sulfate | 33% |
| Zinc Oxide | 15% |
| Zinc Undecylenate | 25% |
| Corn Starch | 9% |
| Kaolin | 9% |
| Silicone Dioxide | 9% |

In each of the above examples, the ingredients are mixed in a blender. More specifically, all of the ingredients are combined in a large vat and blended at low to medium speed until all of the ingredients are completely blended to produce a powder mixture. In one preferred embodiment, each batch totals 2,200 pounds with each of the ingredients present according to the various percentages set forth in the above examples. The resultant powder mixture is filled within bottles containing 141.75 grams of the powder composition. Smaller bottles containing 60 grams of the composition may also be used for sample purposes.

In use, the powder composition is sprinkled in footwear or within the sockets or other skin engaging surfaces of prosthetic attachments and fittings on a daily basis for seven days. After the first week, the powder composition is applied only once per week as a regular regime to prevent future bacterial growth.

While the composition of the present invention has been described in accordance with various preferred and practical embodiments thereof, it is recognized that departures from the instant disclosure are contemplated within the spirit and scope of the present invention.

What is claimed is:

1. An odor eliminating powder composition comprising:
aluminum potassium sulfate in an amount of between 22% and 62% by weight of the composition;
zinc oxide in an amount of between 8% and 36% by weight of the composition; and
zinc undecylenate in an amount of between 6% and 34% by weight of the composition.

2. The composition as recited in claim 1 further comprising:
Kaolin in an amount of between 3% and 28% by weight of the composition.

3. The composition as recited in claim 1 further comprising:
silicone dioxide in an amount of between 0.5% and 15% by weight of the composition.

4. The composition as recited in claim 1 further comprising:
talc in the amount of between 1% and 18% by weight of the composition.

5. The composition as recited in claim 1 further comprising:
corn starch in an amount of between 1% and 18% by weight of the composition.

6. An odor eliminating powder composition comprising:
aluminium potassium sulfate in an amount of between 22% and 62% by weight of the composition;
zinc oxide in an amount of between 8% and 36% by weight of the composition;
zinc undecylenate in an amount of between 6% and 34% by weight of the composition;
Kaolin in an amount of between 2% and 28% by weight of the composition; and
silicone dioxide in an amount of between 0.5% and 15% by weight of the composition.

7. The composition as recited in claim 6 further comprising:
talc in an amount of between 1% and 18% by weight of the composition.

8. The composition as recited in claim 6 further comprising:
corn starch in an amount of between 1% and 18% by weight of the composition.
